(19) United States
(12) Patent Application Publication
(10) Pub. No.: US 2024/0165423 A1
(43) Pub. Date: May 23, 2024

(54) METHODS, SYSTEMS AND DEVICES FOR MODULATING CENTRAL NERVOUS SYSTEM INFLAMMATION AND POST-OPERATIVE PAIN

(71) Applicant: Arizona Board of Regents on Behalf of the University of Arizona, Tucson, AZ (US)

(72) Inventors: Mohab M. Ibrahim, Tucson, AZ (US); Laurent Martin, Tucson, AZ (US); Marvin J. Slepian, Tucson, AZ (US)

(21) Appl. No.: 18/551,727

(22) PCT Filed: Mar. 22, 2022
(86) PCT No.: PCT/US2022/021336

§ 371 Date: Sep. 21, 2023

Related U.S. Application Data
(60) Provisional application No. 63/164,386, filed on Mar. 22, 2021.

(51) Int. Cl. A61N 5/06

(52) U.S. Cl. CPC A61N 5/0622; A61N 2005/0648; A61N 2005/0663

(57) ABSTRACT

Methods, systems and devices are disclosed that provide, among other benefits, an adequate and effective pain management that can be implemented as part of surgical planning, pain management, and other applications. An example methodology for reducing post-operative pain of a subject includes administering, daily, for a first number of days before a surgery on the subject and according to a first pre-arranged schedule of administrations, light with one or more wavelengths in the range between approximately 515 nm and approximately 535 nm to the subject. The method further includes administering, daily, for a second number of days following the surgery and according to a second pre-arranged schedule of administrations, light with one or more wavelengths in the range between approximately 515 nm and approximately 535 nm. The light is administered for a time period during each day of the light administration.

### CROSS-REFERENCE TO RELATED APPLICATION(S)

This application claims priority to the provisional application with Ser. No. 63/164,386 titled “METHODS, SYSTEMS AND DEVICES FOR MODULATING CENTRAL NERVOUS SYSTEM INFLAMMATION AND POST-OPERATIVE PAIN,” filed Mar. 22, 2021. The entire contents of the above noted provisional application are incorporated by reference as part of the disclosure of this document.

### STATEMENT REGARDING FEDERALLY SPONSORED RESEARCH

This invention was made with government support under Grant No. AT009716 awarded by the National Institutes of Health. The government has certain rights in the invention.

### TECHNICAL FIELD

The disclosed technology relates to using light sources to manage or reduce pain and inflammation in a subject.

### BACKGROUND

Postoperative pain severely impacts quality of life and functionality, especially in the elderly. While current pharmacologic methods of pain management are effective in many cases, the associated side effects can limit their use. Non-pharmacological methods would minimize drug reliance, facilitating a reduction of the opioid burden. Green light therapy has been shown to be effective in reducing chronic pain in both humans and rodents. However, there is still a need for developing more effective pain management techniques that can be applied to post operative care, as well as alleviating inflammation of the nervous system.

### SUMMARY

The disclosed embodiments relate to methods, systems and devices, which among other features and benefits, provide an effective pain control methodology that can be implemented as part of surgical planning, pain management, and other applications. In one example embodiment, a method for reducing post-operative pain of a subject is described. The method includes administering, daily, for a first number of days before a surgery on the subject and according to a first pre-arranged schedule of administrations, light with one or more wavelengths in the range between approximately 515 nm and approximately 535 nm to a retina of the subject. The method further includes administering, daily, for a second number of days following the surgery and according to a second pre-arranged schedule of administrations, light with one or more wavelengths in the range between approximately 515 nm and approximately 535 nm to the retina of the subject. The light is administered for a time period during each day of the light administration.

### BRIEF DESCRIPTION OF THE DRAWINGS

FIG. 1 illustrates the difference in thermal pain sensitivity after exposure to different intensity of green light in example experiments.

FIG. 2 illustrates the procedure and results of exposure to green light from green light emitting diodes (GLEDs) and white light from white light emitting diodes (WELDs) based on an example experiment.

FIG. 3 illustrates a table listing of the antibodies used in the experiments.

FIG. 4 illustrates the procedure results of exposure to green light from GLEDs and white light from WLEDs, administered in complement of commonly used pain medication, based on another example experiment.

FIG. 5 illustrates the procedure and results of exposure to green light from GLEDs and white light from WLEDs, administered in complement of commonly used pain medication, based on yet another example experiment.

FIG. 6 illustrates endogenous opioid levels and receptor expression in elderly male rats after exposure to WLED light or GLED light.

FIG. 7 illustrates results of synaptic expression of glutamatergic receptors in elderly male rats exposed GLED and WLED light in an example experiment.

FIG. 8 illustrates inflammation-involved cytokine levels after exposing elderly male rats to GLED in an example experiment.

FIG. 9 illustrates microglial activation in elderly male rats after surgery exposed to GLED light in an example experiment.

FIG. 10 illustrates a set of operations that can be carried for reducing post-operative pain of a subject according to an example embodiment.

FIG. 11 illustrates a set of operations that can be carried for enhancing pain sensitivity of a subject according to an example embodiment.

FIG. 12 illustrates a set of operations that can be carried for reducing microglial activation according to an example embodiment.

FIG. 13 illustrates example sections of the dorsal horn of the spinal cord of rats exposed to WLED and GLED.

FIG. 14 illustrates a set of operations that can be carried for reducing astrogliosis according to an example embodiment.

FIG. 15 illustrates a schematic diagram of an example device that can be used as part of the disclosed embodiments.

### DETAILED DESCRIPTION

In this patent document, we demonstrate, based on experiments conducted on rats, that green light exposure reduced post-surgical hypersensitivity. Moreover, this therapy potentiated the opioid antinociceptive effect on mechanical allodynia in both sexes. Nevertheless, opioid-induced relief of thermal hyperalgesia was not potentiated by green light exposure in females. Through biochemical approaches and 3D-modeling of microglia, we showed that green light exposure can increase endogenous opioid levels while lessening synaptic plasticity and neuroinflammation in the spinal cord collected from male rats. Importantly, this patent document presents new concepts about how light exposure can affect central nervous system inflammation and plasticity. Clinical translation of these results can provide patients with improved pain control, can decrease opioid consumption, and given the noninvasive nature of green light, this innovative therapy is readily implementable in hospitals.

In the patent document, the terms green light emitting diode (GLED) and white light emitting diode (WLED) are used as example illumination sources for green light and white light, respectively. These terms are also sometimes used to indicate illumination from, or exposure to, green light and white light, respectively.

The number of surgical cases performed around the world is on the rise, with more than 313 million cases in 2012. In the US, over 30% of surgeries are performed on patients older than 64, and a significant portion of these surgeries are done on elective or planned basis. Managing postoperative pain in elderly patients is challenging. Elderly patients present comorbidities and changes in pharmacodynamic and pharmacokinetic. Additionally, patients with chronic pain present a greater challenge for post-operative pain control and require greater amounts of opioids. Patients with uncontrolled post-operative pain may experience increased hospital stays with physiological and psychological comorbidities. Therefore, an adequate and effective post-operative pain control method is essential for surgical planning.

There are three main contributors to post-surgical pain severity. First, more severe pain is associated with pre-existing pain condition, chronic opioid use, and surgical procedure to be performed. Second, altered afferent input results in hypersensitivity of tactile stimuli. Third, injuries promote release of inflammatory mediators which decrease pain threshold, promote spontaneous activity and enlarge receptive fields of nociceptors. Cytokines also affect central sensitization. In rodents, tumor necrosis factor-alpha (TNF-α) increases the activities of N-methyl-D-aspartate (NMDA) and α-amino-3-hydroxy-5-methyl-4-isoxazolepropionic acid (AMPA) receptors, which contribute to increased surgical pain. Therefore, inflammatory and plasticity mechanisms are key regulators of post-surgical pain.

Opioids are one of the most commonly prescribed medications for acute post-surgical pain treatment. However, many side effects are associated even with short term use of opioids; i.e. delirium, sedation, nausea, and decreased respiratory rate. In the elderly population, these side effects are even more pronounced, which may lead to decreased mobility, reduced independence and potential morbidity. Collectively, this contributes to ‘frailty’ of the elderly and is further compounded by inadequate pain management. Therefore, a new strategy is needed to decrease post-operative pain for elective surgical cases, particularly in the elderly. The reduction of post-surgical pain may lead to a reduction in the use of opioids, with accompanied decreased side effects.

Different colors of light alter nociception and provide significant relief in both rodents and humans. A pre-clinical study conducted in rodents suggests that green light emitting diode (GLED) induces analgesia in both acute and chronic pain models. Results demonstrated an increase in endogenous opioid mRNAs, though the mechanisms remain unknown. On the other hand, exposing rats to red light-emitting diodes induces thermal and mechanical hypersensitivity in a rodent injury-free model. These pre-clinical studies thus identified a heretofore unknown light effect by which different wavelengths can affect pain sensitivity. In the present patent document, we examine potential underlying mechanisms of GLED therapy and assess its benefits in managing post-surgical pain in the elderly as an example application. Our central hypothesis is that GLED will reverse postoperative thermal and mechanical hypersensitivity in rats by acting on the endogenous opioid system and by modulating inflammatory and plasticity processes.

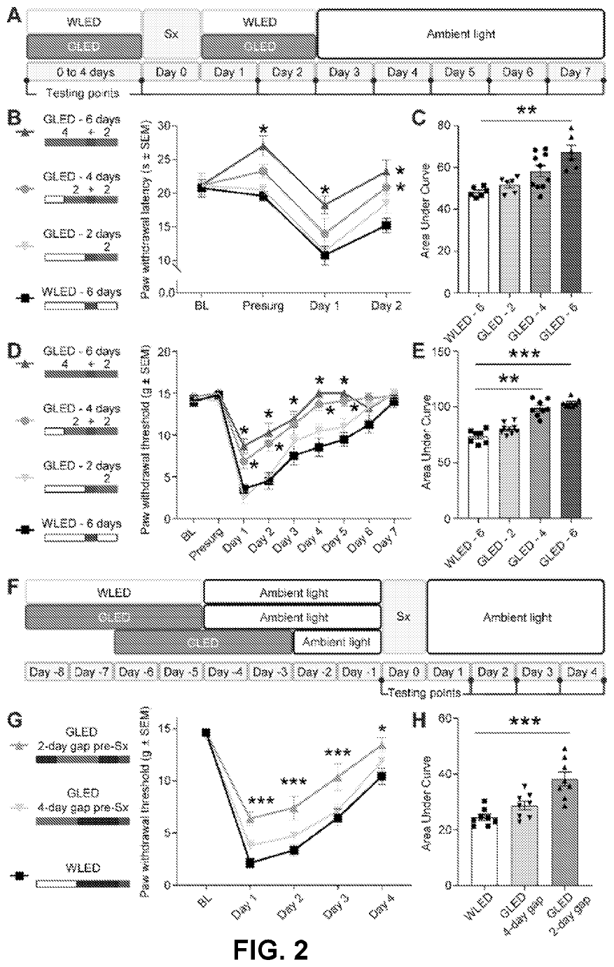

The following types of animals were used in the studies described below. Specific pathogen-free, elderly male Sprague-Dawley rats (>15 months, male weight at testing 600-650 g, female weight at testing 350-390 g; Harlan-Sprague-Dawley, Indianapolis, IN) were housed in climate-controlled rooms on a 12-h light/dark cycle and were allowed to have food and water ad libitum. All procedures were approved by the University of Arizona Animal Care and Use Committee and conform to the guidelines for the use of laboratory animals of the National Institutes of Health (publication no. 80-23, 1966). Key experiments were replicated using a randomized, double-blinded protocol. Randomization, exposure of rats to GLED, behavioral testing, unblinding and data analysis were performed by different individuals. A total of 314 male and 60 female rats were used for this study. Individual panels (e.g., A, B, C, etc.) in the figures are referred to in this patent document using the corresponding figure number. For example, Panel A in FIG. 2 is referred to as FIG. 2A.

For behavioral studies, rats were exposed to light in an isolated room. Only TK was allowed to enter the room. Behavioral testing was conducted in a separated room by LFM. TK brought the rat cages 20 to 30 minutes before behavioral testing. LFM never approached animals while they were being exposed to light. After data acquisition and analysis, results were sent to SMW, who unblinded the study.

For immunohistochemistry and ELISA, neither SMW nor LFM were in contact with the animals while they were being exposed to light. TK brought number-labeled rats from the exposure room to the dissecting room. LFM and SMW collected samples, unaware of the conditions. After ELISA analysis and modeling of microglia, results were sent to KC, who unblinded the study.

For drug injections, rats were physically restrained, and a tent of skin was pulled taut to separate the epidermal and dermal layers from the musculature. A 25-gauge needle was inserted posteriorly between the animal scapulas into the subcutaneous space at a 45° angle to the skin. Saline solution or two different morphine sulfate doses were injected (1 mg/kg or 5 mg/kg, diluted in 0.9% saline, Sigma Aldrich, #M8777). The doses of medications chosen were based on previously published data in animals.

To induce acute post-surgical pain, rats were subjected to an incisional surgery. Briefly, rats were anesthetized with 2% isoflurane in O2 anesthesia (total time under anesthesia was <10 minutes). After scrubbing the left hind paw with chlorhexidine and 70% ethanol, a 1 cm long incision, from the heel toward the toes on the plantar aspect of the left hind paw, was made through skin and fascia, exposing the underlying muscle. The flexor muscle was then elevated and longitudinally incised, leaving the muscle origin and insertion intact. After hemostasis with gentle pressure, the skin was closed with two mattress sutures using 5-0 nylon on a curved needle.

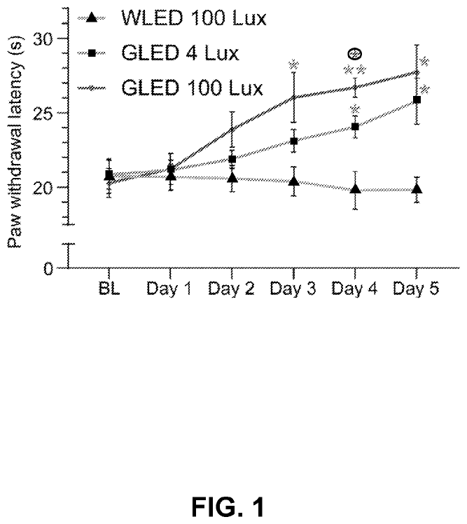

All visible spectrum LED flex strips were purchased from ledsupply.com (VT, USA). The specifications of the LEDs were: (i) #LS-AC50-GR-006, 525 nanometer wavelength (i.e., green), 8 watts, 120 Volts, 120 degree beam angle; and (ii) #LS-AC50-WW-006, white, 9.6 watts, 120 Volts, 120 degree beam angle. LED strips were affixed on the top of racks where rats were housed, allowing global diffusion of light. Rats were exposed to the various LED in these cages with full access to food and water in a dark room devoid of any other source of light. Following behavioral assessment, the rats were returned to their cages for additional LED exposure. At the end of daily testing, the rats were returned to their regular animal room where they were exposed to room light illuminated with Sylvania Octron 3500K F032/835 model which is 48″ in length and power output of 32-Watt florescent bulbs producing intensity of 750 Lux. A Lux meter (Tondaj LX1010B, Amazon.com) was used to determine the illuminance and luminous emittance of the LED strips. We chose different times and intensities of exposure to characterize the antinociceptive effect of GLED (FIGS. 1 and 2). For our main experiments, we opted for an exposure of 4 days, based on our own observations. As peak pain behavior is observed in the two days following surgery, we opted to cover this period with green light exposure. Hence, our study design for all experiments described in this patent document corresponds to an exposure of 2 days before and 2 days after surgery for a total of 4 days of green light exposure, unless indicated otherwise. Rats were exposed to green light 8 hours per day, from 7 am to 3 pm.

Paw withdrawal latencies were determined in the following manner. Rats were acclimated within Plexiglas enclosures on a clear glass plate maintained at room temperature. A radiant heat source (high-intensity projector lamp) was focused onto the plantar surface of the hind paw. When the paw was withdrawn, a motion detector halted the stimulus and a timer. A maximal cutoff of 33.5 sec was used to prevent tissue damage.

The assessment of tactile sensory thresholds was determined by measuring the withdrawal response to probing the hind paw with a series of calibrated fine (von Frey) filaments. Each filament was applied perpendicularly to the plantar surface of the paw of rats held in suspended wire mesh cages. Primary hyperalgesia was evaluated in accordance to previously described methods. Withdrawal threshold was determined by sequentially increasing and decreasing the stimulus strength (the “up and down” method), and data were analyzed with the nonparametric method of Dixon, as described by Chaplan and colleagues and expressed as the mean withdrawal threshold.

Spinal cords were harvested by hydraulic extrusion from elderly Sprague-Dawley male rats, and tissue lysates were generated by homogenization and trituration in lysis buffer (50 mM Tris-HCl, 150 mM NaCl, 1% Triton-X-100, 0.5% sodium deoxycholate, 1 mM EDTA, 0.1% SDS, pH 7.4). To isolate ipsilateral dorsal horns, the lumbar part (L4-L6) of the spinal cord was hemisected prior to homogenization. Lysis buffer was supplemented with protease inhibitor cocktail (Bimake, B14002), phosphatase inhibitors (Bimake, B15002), and Pierce universal nuclease (Fisher Scientific, PI88701). Protein concentrations were determined using BCA protein assay (ThermoFisher Scientific, PI23225).

Elderly male rats were sacrificed by isoflurane overdose followed by decapitation. Lumbar spinal cords were collected by hydraulic extrusion and the ipsilateral part of the lumbar dorsal horn was then dissected. The synaptic fractionation protocol was adapted from a previously described method. In brief, samples were homogenized in ice-cold buffer (sucrose 0.32 M, HEPES 10 mM, pH 7.4) and then centrifuged at 1000 g for 10 minutes to remove nuclei and large debris. The remaining supernatants were centrifuged at 12,000 g 20 min to obtain a crude membrane fraction. Pellets were resuspended in hypotonic EDTA buffer (4 mM HEPES, 1 mM EDTA, pH 7.4) to chelate calcium, and centrifuged at 12,000 g for 20 min to pellet the synaptosomal fractions. Synaptosomes were then incubated in a low-triton buffer (20 mM HEPES, 100 mM NaCl, 0.5% Triton X, pH 7.2) for 15 min on ice and centrifuged at 12,000×g for 20 min at 4° C. The supernatant contained the presynaptic membrane fraction, referred to as the triton-soluble fraction. The resulting pellets were further extracted with a high-detergent buffer (20 mM HEPES, 0.15 mM NaCl, 1% triton X100, 1% deoxycholic acid, 1% SDS, pH 7.5) for 1 hour and then centrifuged for 15 minutes at 10,000 g to obtain the postsynaptic density fraction remaining in supernatant. The enrichment quality of each fractions was assessed by immunoblotting for PSD95, a postsynaptic-specific structure, and synaptophysin, which is not found in the postsynaptic element. All buffers were supplemented with nuclease, protease and phosphatase inhibitor cocktails. BCA protein assay was used to analyze protein concentrations.

Rat ELISA were purchased from MyBioSource (San Diego, CA) to measure serum, CSF, and spinal cord lysate levels of endogenous opioids (β-endorphin, #MBS452166; Proenkephalin, #MBS726498 and Dynorphin, #MBS720677). ELISA for IL-10 were purchased from RayBiotech (#ELR-IL10-1), and for TNFα from R&D (#RTA00). Procedures were conducted according to the manufacturers' instructions. Colorimetric detection was based on H2O2/TMB reaction. To determine the optical density of each well, a Biotek Epoch microplate reader was set to 450 nm. Determination of endogenous opioid and cytokine levels involved triplicate determinations for each sample. For lysate samples, each concentration was normalized to the weight of the different spinal cords after collection.

For detection of AMPA and NMDA sub-units, CamKII, Src family kinases, PKCγ, and their respective phosphorylated forms, 10 μg (PSD/Non-PSD fractions) to 40 μg (total lysate) of proteins was loaded into wells of 3-8% gradient SDS-PAGE gels (Criterion XT, Biorad). Gels were transferred to 00.25 PVDF membranes after activation in 100% methanol. Membranes were immunoblotted with antibodies described in table 1 in Appendix A, after a blocking step for 45 minutes in Tris-buffered saline, 0.1% Tween, 5% milk or BSA, pH 7.6. Membranes were then incubated overnight with antibodies diluted in Tris-buffered saline, 0.1% Tween, pH 7.6, with 2% milk for total protein or 2% BSA for phosphorylated protein detection. Goat anti-rabbit HRP or Goat anti-mouse HRP (1/10000 or 1/20000, Jackson Immunoresearch; room temperature, 2 h) were used as secondary antibodies. Protein bands were detected with Azure Sapphire Biomolecular Imager (Azure Biosystems) after applying chemiluminescent reagent for 2 minutes (ThermoFisher Scientific). Bands were quantified densitometrically with Image J software (National Institutes of Health). Detection of PSD95 and Synaptophysin proteins was used as control for fractionation protocol, and actin protein levels were quantified in total lysates for loading control.

Animal perfusion and spinal cord collection: to collect spinal cords from Sprague-Dawley male rats, animals were anesthetized with 1-5% isoflurane delivered in 02 and then perfused with phosphate-buffered saline (PBS) and 4% paraformaldehyde (PFA). Single 4 cm lateral incision was made through integument and abdominal wall just beneath rib cage. A cut was then made through rib cage up to the clavicle. A 25-gauge needle was inserted through left ventricle into ascending aorta. Approximately 60 mL of PBS and 150 mL of 4% PFA were injected through the heart at a steady flow rate. After fixation, rats were decapitated and lumbar spinal cords were collected by hydraulic extrusion. Spinal cords were postfixed in 4% PFA for 2-3 hours and then immersed in 30% sucrose for 24 hours. Spinal cords were then flash frozen into Tissue-Tek and stored at −80° C. 15 μm frozen sections were sliced using Cryostar NX50.

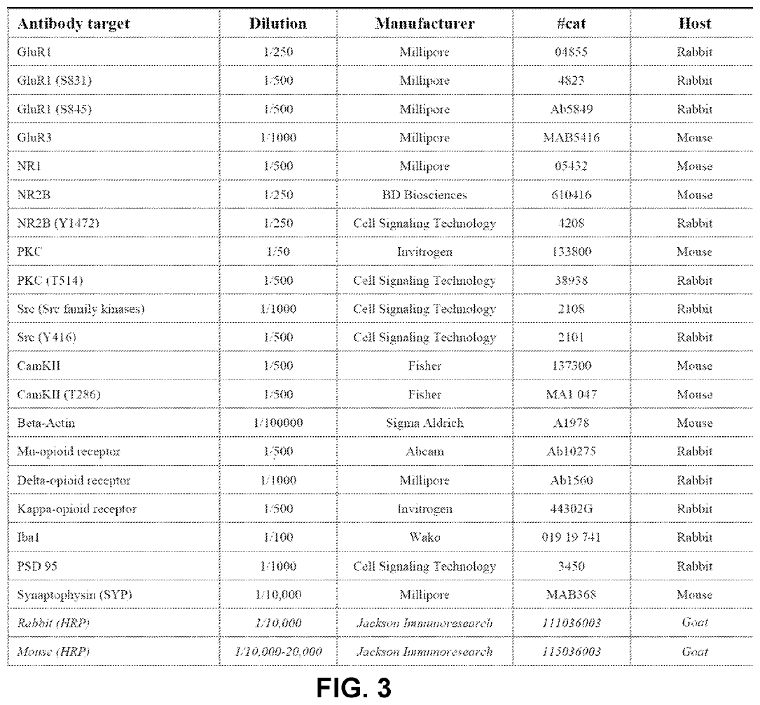

Microglia immunostainings: briefly, slices were permeabilized in 0.3% Triton-X-100 with 1% BSA diluted in PBS, and proteins of interest were stained overnight at 4° C. in the same buffer with antibodies directed against Iba1 (FIG. 3). After primary probing, slices were rinsed and incubated for 2 hours at room temperature with appropriate fluorescent conjugated secondary antibodies (Jackson Immunoresearch). A DAPI counterstaining was performed after secondary antibody incubation, and slices were mounted with FluorSave reagent (Millipore #345789).

Microglia images were obtained with a Zeiss LSM 880 confocal microscope. Identical acquisition parameters for all conditions were used. Each slide was randomly encoded to run all the analysis processes in a blindfolded way. Images were obtained with a Plan-Apochromat 63×/1.40 Oil objective. All planes of the same image were acquired with a 400 nm Z step, in the laminae I and II from the dorsal horns of elderly male rats that underwent paw incision surgery (ipsilateral region). Experimenter was blinded to the condition, and selected one to two microglia per slices, as long as their processes and morphology were not damaged by freezing and cutting steps. Laser power and gain were adapted to obtain the best signal without saturation. Microglia morphology was analyzed with Imaris software (Bitplane). Briefly, microglial processes were selected and modelled with filament tracer. Cell somas were selected and modelled with the Volume tool. All thresholds were automatically selected by software. A total of 26 microglia from 3 independent experiments were analyzed. All parameters for each microglia were automatically measured by the software and then exported to an Excel file for further analysis. Branch orders correspond to the level of process ramification: branches of the first order correspond to the processes between somas and first ramification points, whereas branches of the tenth order correspond to the processes between the ninth and tenth ramification points. To reflect microglial complexity, we analyzed the percentages of each branch order and compared them between our two conditions.

All data were expressed as mean±standard error of the mean (SEM), unless stated otherwise. Statistical analysis was run using GraphPad Prism software 8.0 (San Diego, CA). All data were first tested for Gaussian distribution and heteroscedasticity, respectively using Shapiro-Wilk and Barlett's tests. The statistical significance of differences between mean was determined by parametric and non-parametric analysis followed by post-hoc comparisons. Differences were considered to be significant if the probability value p≤0.05. No outlier data were removed. All data were plotted using Prism software.

GLED potentiates morphine reversal of postoperative thermal hypersensitivity in male rats and mechanical hypersensitivity in both male and female rats. We previously reported that GLED (525-535 nm wavelength) for 8 hours a day for 5 days demonstrated reversal of hypersensitivity in a chronic pain model. Managing pain in the elderly population is challenging, thus we explored the potential benefits of green light exposure in a model post-operative pain in rats. Two different intensity levels of GLED (4 and 100 Lux) were compared, with n=6-7, mean±SEM, a two-way ANOVA followed by Tukey's posthoc test. In FIG. 1, gray asterisks illustrate significant differences to saline control, and the circle asterisk illustrates significant difference to 4 Lux condition, *p<0.05, **p<0.01. Increasing GLED intensity produced a faster decrease in thermal pain sensitivity. Given that analgesia was more pronounced when we exposed rats to 100 Lux compared to 4 Lux (see FIG. 1), we decided to use 100 Lux intensity exposure for testing behavioral and biological effects of white and green LED on elderly rats. To identify the exposure protocol that provides best results, we first exposed elderly rats that had undergone incisional surgery of the hind paw to different durations of pre- and post-surgical GLED exposure (see FIG. 2A). More specifically, old male rats (>600 g) were exposed to green LED (λ=525 nm) or white LED (8 h/day, 100 Lux) for 5 days, and tested each day to evaluate thermal sensitivity.

Data demonstrated that the antinociceptive effects of GLED on both thermal and mechanical hypersensitivity are dependent on the duration of GLED exposure (FIGS. 2B-2E). Furthermore, increasing the gap between exposure and surgery revealed that GLED provides long-lasting antinociceptive effect (FIGS. 2F-2H). As peak pain behavior is observed in the two days following surgery, we opted to cover this period with green light exposure. Based on these preliminary results (FIGS. 1 and 2), our main study design corresponds to an exposure of 2 days before and 2 days after surgery for a total of 4 days of green light exposure. In particular, in FIG. 2A, experimental design for thermal and mechanical hypersensitivity assessment, depending on time of exposure, are illustrated. Old male rats (>600 g) were exposed to green LED (GLED, λ=525 nm; 0, 2, or 4 days, 8 h/day, 100 Lux) or white LED (WLED; 4 days, 8 h/day, 100 Lux) prior to paw incision surgery (Sx), and then GLED or WLED for 2 days post-surgery. FIG. 2B illustrates that following measurement of baseline (BL), thermal hypersensitivity was assessed before (Presurg) and two days after surgery (n=6-10, mean±SEM, two-way ANOVA followed by Tukey's posthoc test; asterisks illustrate significant differences with white condition of the same day, *p<0.05). GLED resulted in reversing thermal hypersensitivity. FIG. 2C shows area under the curve analyses for FIG. 2B, corresponding to paw withdrawal latencies among different durations of GLED and WLED exposure (n=6-10, mean±SEM, Kruskal Wallis test followed by Dunn's posthoc test, **p<0.01). The results show GLED reversal of thermal hypersensitivity depends on exposure duration. In FIG. 2D, mechanical hypersensitivity was assessed before light exposure, before surgery, and for 7 days after surgery (n=6-10, mean±SEM, two-way ANOVA followed by Tukey's posthoc test, black asterisks illustrate significant differences with white condition of the same day, *p<0.05). GLED resulted in reversal of mechanical hypersensitivity. FIG. 2E illustrates the area under the curve analyses for FIG. 2D, corresponding to paw withdrawal threshold among different durations of GLED exposure and WLED exposure (n=6-10, mean±SEM, Kruskal Wallis test followed by Dunn's posthoc test, **p<0.01, ***p<0.001). The results indicate GLED reversal of mechanical hypersensitivity depends on exposure duration. FIG. 2F shows the experimental design for mechanical hypersensitivity assessment, with different gaps prior surgery. Rats were exposed to either 4 days of WLED or GLED before surgery, with a 2- or 4-day gap between exposure termination and surgery. FIG. 2G illustrates the plots when the mechanical hypersensitivity was assessed before surgery and for 4 days after surgery (n=8, mean±SEM, two-way ANOVA followed by Tukey's posthoc test, black asterisks illustrate significant differences with white condition of the same day, *p<0.05, ***p<0.001). FIG. 2H shows the area under the curve analyses for FIG. 2G, representing paw withdrawal threshold among different post-exposure gaps before surgery (n=8, mean±SEM, Kruskal Wallis test followed by Dunn's posthoc test, ***p<0.001). The results indicate that GLED exposure resulted in a long-lasting antinociceptive effect. FIG. 3 illustrates a table listing of the antibodies used in the study.

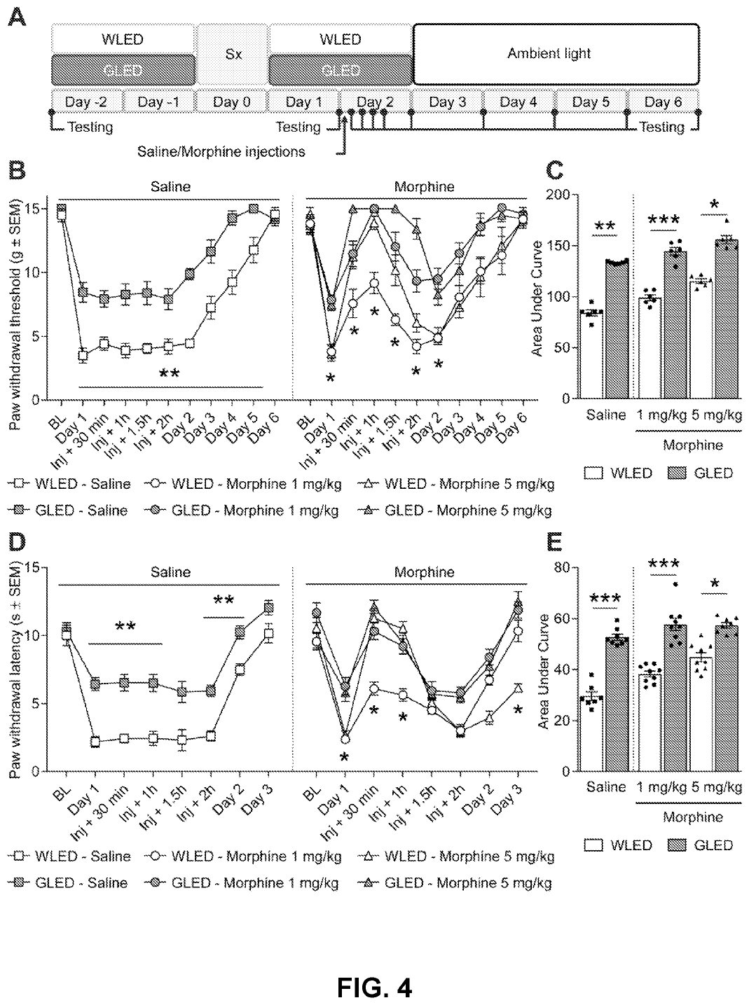

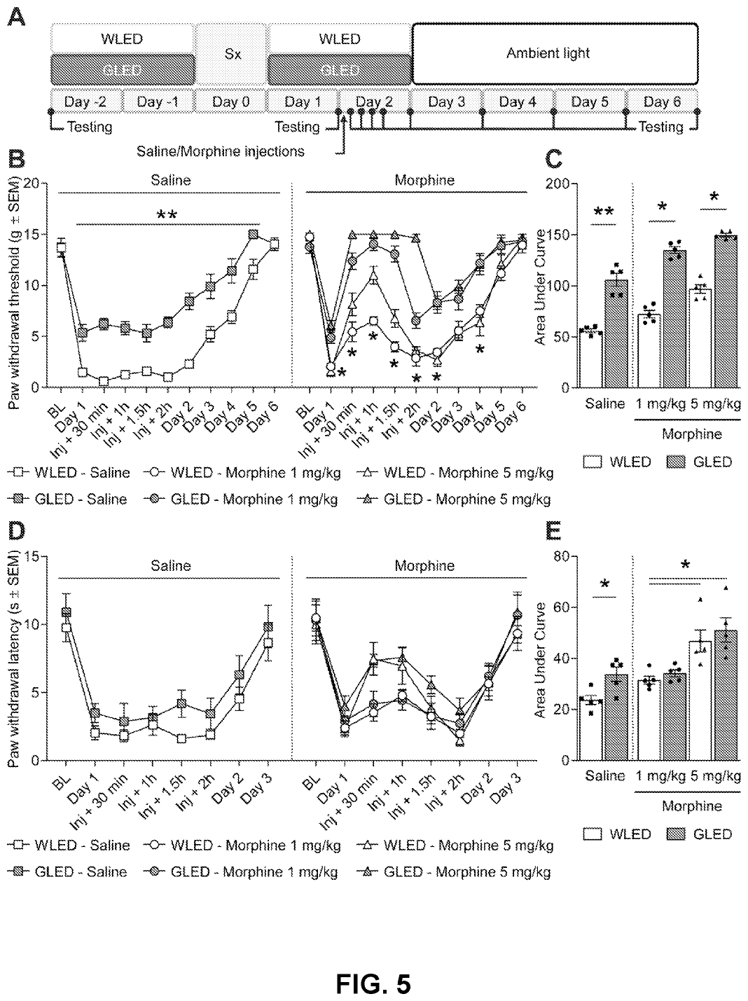

Opioids are one of the most commonly prescribed medications for postoperative pain, and our previous study revealed increase in PENK mRNA expression after exposure to green light. Hence, we sought to investigate their potential synergistic effect. As nociceptive signals are not processed the same way between males and females, we conducted our main experiment in both sexes; male (FIG. 4A) and female (FIG. 5A) elderly rats were exposed to GLED or WLED (white light-emitting diodes) for 2 days prior and 2 days after surgery. 24 hours after surgery, rats were injected subcutaneously with saline or two doses of morphine solution (1 or 5 mg/kg). Mechanical and thermal hypersensitivity were assessed over 3 (thermal) or 6 days (mechanical), and immediately following saline or morphine injection, every 30 minutes over 2 hours.

FIG. 4 illustrates some of the results of exposure to green light from green light emitting diodes (GLEDs) and shows that green light exposure potentiates morphine reversal of postoperative thermal and mechanical hypersensitivity in male rats. FIG. 4A (i.e., FIG. 4, Panel A) shows the experimental design used for mechanical and thermal hypersensitivity assessment, to examine potential synergy between GLED and morphine reversal of postoperative hypersensitivity. Following measurement of baseline (BL), elderly male rats (>600 g) were exposed to white led (WLED) or green LED (GLED), 100 Lux 8 h/day, for two days before paw incision surgery (Sx) and two days after surgery. Saline or morphine solution (1 or 5 mg/kg) was injected subcutaneously 24 h after surgery. FIG. 4B shows that mechanical hypersensitivity was assessed before light exposure, before morphine/saline injection (inj), every 0.5 h after injection until 2 h, and daily until 6 days post-surgery (n=6, mean±SEM, two-way ANOVA, followed by Tukey's posthoc). GLED exposure resulted in potentiation of morphine reversal of postoperative mechanical hypersensitivity. FIG. 4C shows results of the area under the curve analyses for paw withdrawal thresholds presented in FIG. 4B, among saline- and morphine-injected animals exposed to either WLED or GLED (n=6, mean±SEM, Mann Whitney (saline conditions) or Kruskal Wallis test followed by Dunn's posthoc test (morphine conditions), *p<0.05, **p<0.01, ***p<0.001). FIG. 4D shows that thermal hypersensitivity was assessed before light exposure, before morphine/saline injection, every 0.5 h after injection until 2 h, and daily until 3 days post-surgery (n=7-9, mean±SEM, two-way ANOVA, followed by Tukey's posthoc). GLED exposure resulted in potentiation of morphine reversal of postoperative thermal hypersensitivity. FIG. 4E illustrates results of the area under the curve analyses for paw withdrawal latencies presented in FIG. 4D, among saline- and morphine-injected animals exposed to either WLED or GLED (n=7-9, mean±SEM, Mann Whitney (saline conditions) or Kruskal Wallis test followed by Dunn's posthoc test (morphine conditions), *p<0.05, ***p<0.001).

FIG. 5 illustrates additional results of exposure to green light from GLEDs and shows that green light exposure potentiates morphine reversal of postoperative mechanical hypersensitivity in female rats. FIG. 5A shows the experimental design used for mechanical and thermal hypersensitivity assessment, to examine potential synergy between GLED and morphine reversal of postoperative hypersensitivity. Following measurement of baseline (BL), elderly female rats (>350 g) were exposed to white led (WLED) or green LED (GLED), 100 Lux 8 h/day, for two days before paw incision surgery (Sx) and two days after surgery. Saline or morphine solution (1 or 5 mg/kg) was injected subcutaneously 24 h after surgery. FIG. 5B show that mechanical hypersensitivity was assessed before light exposure, before morphine/saline injection, every 0.5 h after injection until 2 h, and daily until six days post-surgery (n=5, mean±SEM, two-way ANOVA, followed by Tukey's posthoc). GLED exposure resulted in potentiation of morphine reversal of postoperative mechanical hypersensitivity. FIG. 5C shows results of the area under the curve analyses for paw withdrawal thresholds presented in FIG. 5B, among saline- and morphine-injected animals exposed to either WLED or GLED (n=5, mean±SEM, Mann Whitney (saline conditions) or Kruskal Wallis test followed by Dunn's posthoc test (morphine conditions), *p<0.05, **p<0.01). FIG. 5D shows that thermal hypersensitivity was assessed before light exposure, before morphine/saline injection, every 0.5 h after injection until 2 h, and daily until three days post-surgery (n=5, mean±SEM, two-way ANOVA, followed by Tukey's posthoc). GLED exposure did not result in potentiation of morphine reversal of postoperative thermal hypersensitivity. FIG. 5E shows results of the area under the curve analyses for paw withdrawal latencies in FIG. 5D, among saline- and morphine-injected animals exposed to either WLED or GLED (n=5, mean±SEM, Mann Whitney (saline conditions) or Kruskal Wallis test followed by Dunn's posthoc test (morphine conditions), *p<0.05).

In both male and female saline-injected groups, GLED exposure yielded significantly higher post-surgery paw withdrawal thresholds from Days 1 to 5, with both GLED and WLED groups returning to baseline values by Day 6 (FIGS. 4B, 5B). Area under the curve analysis showed statistically significant effects of GLED on paw withdrawal threshold when compared to that of the WLED groups (FIG. 4C; FIG. 5C). Paw withdrawal latencies of males in the GLED group were significantly higher from Days 1 to 2 when compared to that of the WLED group, with the exception of the timepoint at 1.5 h post-injection (p=0.051; FIG. 4D). Area under the curve analysis showed statistically significant effects of GLED on paw withdrawal latency when compared to that of the WLED group (FIG. 4E). In females, no significant differences were found between the GLED and WLED groups in paw withdrawal latencies at any time points (FIG. 5D), although area under the curve analyses showed statistically significant trend of GLED on paw withdrawal latency when compared to that of the WLED group (FIG. 5E). These results confirm that GLED reduces hypersensitivity in elderly rats that had received paw incision surgery. This effect seems to be modality-specific, as GLED did not induced thermal anti-hyperalgesia in female rats.

In morphine-injected animals, paw withdrawal thresholds were higher than that of their saline-injected counterparts at up to 1.5 hours post-injection in both males (FIGS. 4B and 5C) and females (FIGS. 4B and 5C). However, only male rats exposed to GLED had significantly higher paw withdrawal latencies (FIGS. 4D and 4E) for both doses of morphine when compared to rats exposed to WLED. Indeed, GLED did not increase paw withdrawal latencies after morphine injection in females (FIGS. 5D and 5E). Importantly, combined GLED exposure and injection of 1 mg/kg of morphine had a similar or greater effect on mechanical hypersensitivity when compared to that of WLED+5 mg/kg morphine. These data collectively show that GLED potentiates the antinociceptive effect of morphine, and that a low dose of morphine used in conjunction with GLED can achieve similar or greater levels of antinociception using a higher dose of morphine by itself.

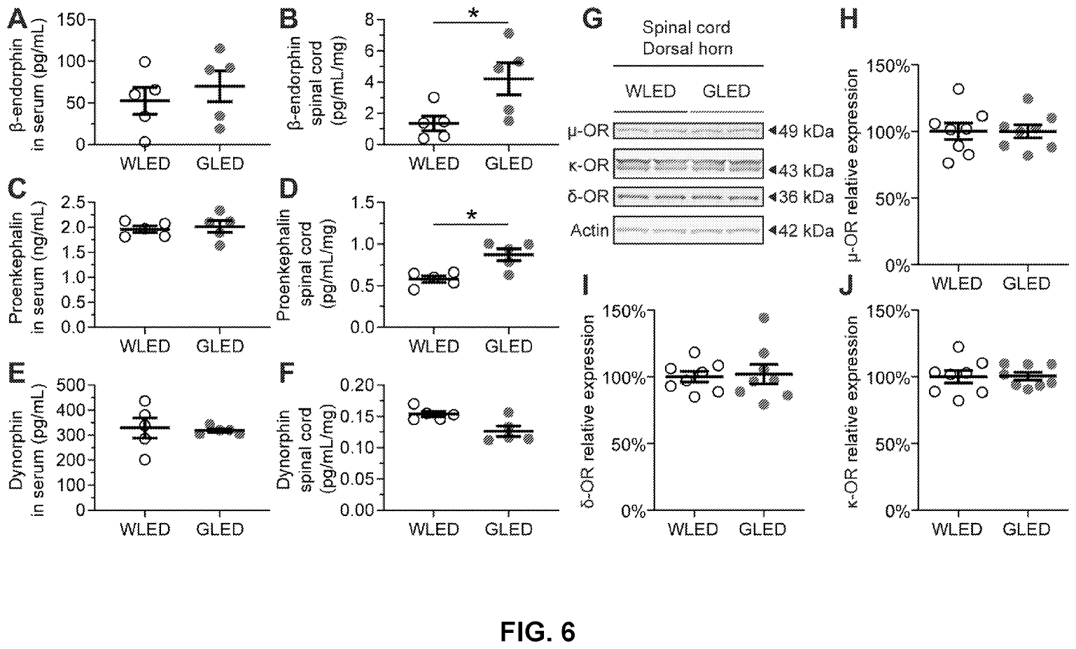

FIG. 6 illustrates endogenous opioid levels and receptor expression in elderly male rats after exposure to white (WLED) light or green LED (GLED) light. Elderly male rats (>600 g) were exposed to green LED (GLED; 2 days; 8 h/day, 100 Lux) or white LED (WLED; 2 days; 8 h/day, 100 Lux) prior to paw incision surgery, and then GLED or WLED for two days post-surgery. Blood and spinal cords were collected at the end of the second day post-surgery. Levels of β-endorphin were analyzed through ELISA assays in both serum (FIG. 6A) and spinal cord lysates (FIG. 6B), as well as Proenkephalin (FIGS. 6C and 6D) and Dynorphin (FIGS. 6E and 6F) (n=5, Mann-Whitney test, *p<0.05). GLED exposure resulted in increased levels of ß-endorphin and Proenkephalin in spinal cord lysates, but not in serum. FIG. 6G shows representative western blots of μ-, δ-, and κ-opioid receptor (OR) expression in the dorsal horn of the lumbar spinal cord after exposure to WLED or GLED. Histogram represents the quantification of μ-OR (FIG. 6H), δ-OR (FIG. 6I), and κ-OR (FIG. 6J) expression (n=8 mean±SEM). No differences in their expression were observed between WLED and GLED conditions.

GLED exposure increases endogenous opioid levels but does not modulate the expression of their receptors in male rats. Naloxone was shown to reverse the antinociceptive effects of GLED, and given that GLED exposure increases PENK mRNA levels and potentiates morphine reversal of postoperative hypersensitivity, we wanted to investigate if GLED exposure had any effect on the endogenous opioid system. Thus, we analyzed endogenous opioid levels and the expression of their receptors in male rats. After surgery, GLED exposure resulted in increased central nervous system (CNS) levels of both β-endorphin and Proenkephalin, without affecting dynorphin levels (FIGS. 6B, 6D, 6F). Importantly, no differences were observed in serum fractions (FIGS. 6A, 6C, 6E), suggesting that GLED-induced antinociception is dependent on central rather than peripheral mechanisms. Elderly rats exposed to green light also did not display any differences in expression of μ-(MOR), δ-(DOR), and κ-opioid receptors (KOR) (FIGS. 6G-6J). These data suggest that reversal of hypersensitivity results, at least in part, of endogenous opioid release.

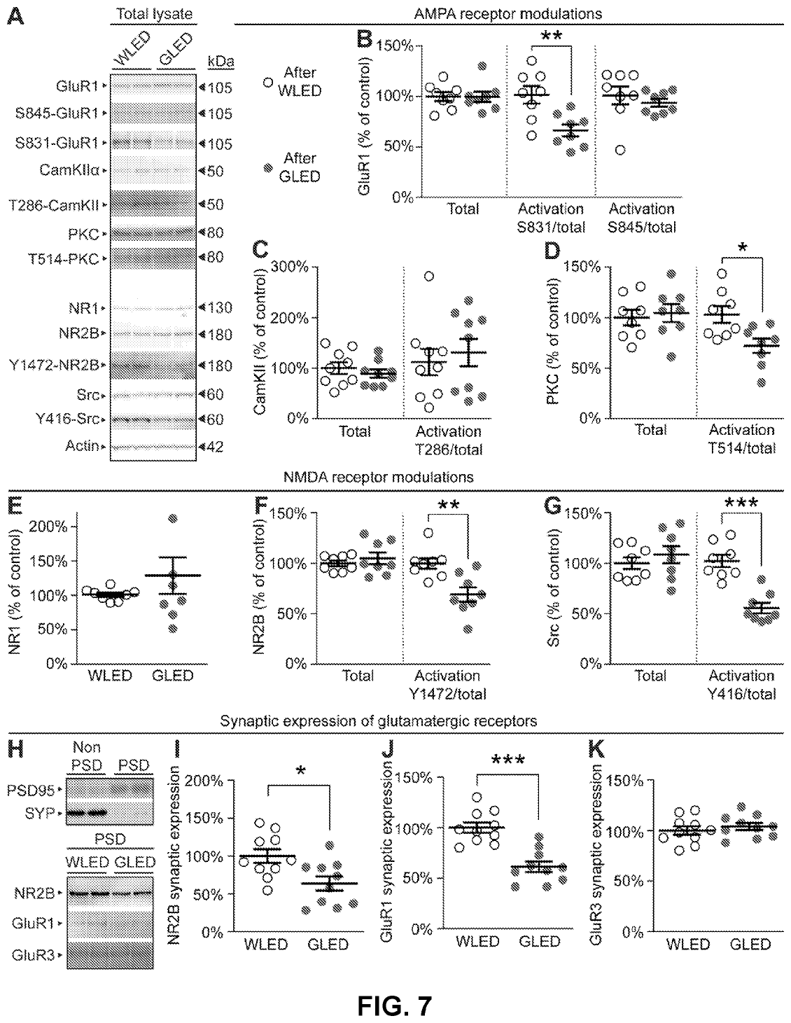

FIG. 7 shows that GLED exposure modulates synaptic expression of glutamatergic receptors in male rats in a model of acute post-surgical pain. Elderly male rats were exposed two days to WLED or GLED before paw incision surgery, and two days after surgery. Spinal cords (dorsal horns) were then collected after rats were exposed to WLED or GLED. FIG. 7A shows example western blots that illustrate expression of glutamatergic receptors and kinases involved in their phosphorylation, as well as their phosphorylated forms. FIG. 7B show a histogram that represents the quantification of GluR1 total expression and its level of phosphorylation on both S831 and S845. (n=8, mean±SEM, Mann Whitney test, **p<0.01). FIG. 7C shows results of quantification of CamKII total expression and its level of phosphorylation on Thr286. (n=9, mean±SEM, Mann Whitney test). FIG. 7D shows results of quantification of PCKy total expression and its level of phosphorylation on Thr514. (n=8, mean±SEM, Mann Whitney test, *p<0.05). FIG. 7E shows results of quantification of NR1 total expression. (n=8, mean=SEM, Mann Whitney test, *p<0.05). FIG. 7F shows results of quantification of NR2B total expression and its level of phosphorylation on Tyr514. (n=8, mean=SEM, Mann Whitney test, **p<0.01). FIG. 7G shows results of quantification of Src family kinase total expression and their level of phosphorylation on Tyr416. (n=8, mean±SEM, Mann Whitney test, ***p<0.001). FIG. 7H shows representative western blots of PSD95, Synaptophysin (SYP), NR2B, GluR1 and Glur3. Western blots illustrate synaptic expression of glutamatergic receptors. Dorsal horns were dissected, and cellular lysates were fractionated to obtain presynaptic and post-synaptic fractions. Expression of PSD95, specific from post-synaptic densities (PSD), and SYP, absent from the PSD, were analyzed to evaluate the enrichment quality. Histogram shows in FIG. 7I represents the quantification of NR2B synaptic expression (n=10, mean=SEM, Mann Whitney test, *p<0.05). Histogram shown in FIG. 7J represents the quantification of GluR1 synaptic expression (n=10, mean±SEM, Mann Whitney test, ***p<0.001). Histogram shown in FIG. 7K represents the quantification of GluR3 synaptic expression (n=10, mean±SEM, Mann Whitney test).

GLED modulates post-synaptic expression of glutamatergic receptors in male rats. GLED exposure resulted in increased levels of endogenous opioids, specifically in the CNS, suggesting that analgesia induced by green light may rely more on central than peripheral mechanisms. We then evaluated GLED potential effect on plasticity, analyzing expression and activation of glutamatergic receptors in the dorsal horn of spinal cords collected from elderly male rats that underwent paw incision surgery. Importantly, AMPA receptors containing GluR1, GluR2 and GluR3 subunits are largely expressed in the dorsal horn, and NR2B-containing receptors dominate synaptic responses in the adult spinal cord. Dorsal horns of lumbar spinal cords were collected from elderly male rats after a 4-day exposure of WLED or GLED. These samples were then lysed and fractionated to obtain pre- and post-synaptic fractions. In total lysates, no significant differences were found in expression of GluR1, NR1 and NR2B (FIGS. 4A-4F). However, phosphorylation of GluR1 serine 831 was significantly decreased, whereas no effects were observed on serine 845 (FIG. 7B). Western blot analysis of NR2B phosphorylation also demonstrated a decreased phosphorylation of NR2B subunit on tyrosine 1472 (FIG. 7F). These results reveal that, after performing surgery on elderly male rats, GLED exposure was able to decrease activation of both NMDA and AMPA receptors, key receptors involved in plasticity modulation in the brain. Many kinases regulate synaptic expression of glutamatergic receptors during synaptic potentiation. Amongst them, CaMKII, PKC, and PKA play a prevalent role in phosphorylating GluR1 subunit, leading to increased synaptic expression and transmission potentiation. Src family kinases (SFKs) are also crucial for NMDA receptors and modulates its trafficking through phosphorylation. Notably, NR2B receptors have increased phosphorylation by PKC and SFKs in the spinal cord from a rodent model of chronic pain. Our results confirmed that GLED-induced plasticity modulation involves these key regulators. Indeed, GLED decreased SFK and PKC activation, thus leading to reduced phosphorylation of NMDA and AMPA receptors (FIGS. 7B and 7F). As glutamatergic receptor phosphorylation controls their trafficking and synaptic expression, we analyzed the synaptic expression of both NMDA and AMPA receptors. By analyzing PSDs content in western blots, we found that both NR2B and GluR1 subunits were less expressed at the synapse following GLED exposure (FIGS. 7H-7K). No effect was observed on GluR3. Overall, these data show neuronal plasticity changes in glutamatergic receptors that suggest decreased glutamatergic transmission contributing to the long-lasting antinociceptive effects of GLED in elderly male rats.

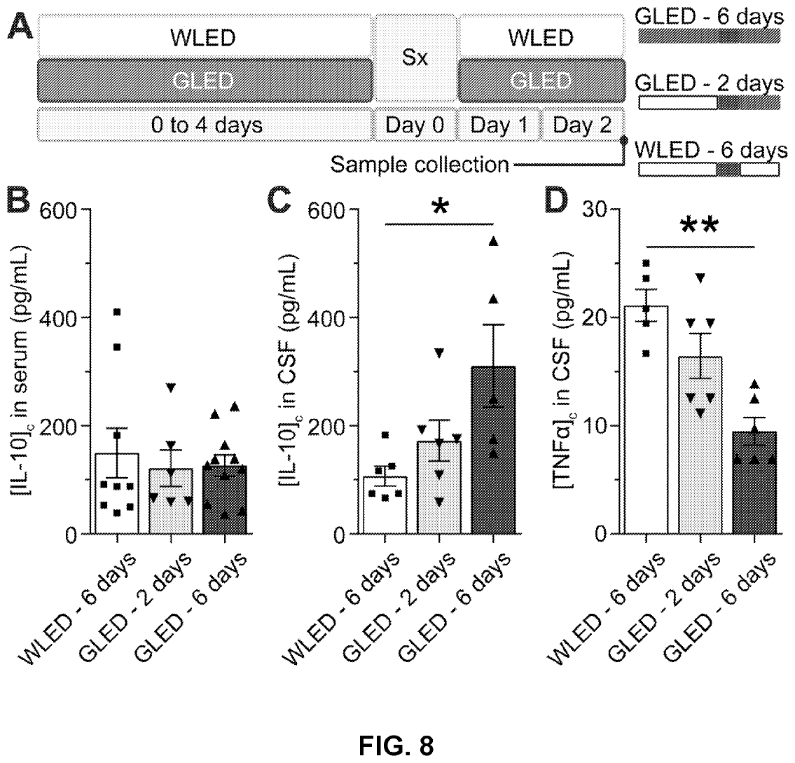

FIG. 8 illustrates inflammation-involved cytokine levels after exposing male rats to GLED. FIG. 8A shows the experimental design that was used to analyze cytokines levels in both serum and cerebrospinal fluid (CSF). Elderly male rats were exposed to green LED (GLED; 0 or 4 days) or white LED (WLED; 4 days), 8 h/day 100 Lux, prior to paw incision surgery (Sx), and then GLED or WLED for two days post-surgery. Blood and cerebrospinal fluid (CSF) were collected at the end of the second day post-surgery. FIG. 8B shows ELISA assay results representing the average IL-10 concentration in serum samples (n=6-11, mean±SEM, Kruskal Wallis test, followed by Dunn's posthoc). FIG. 8C shows ELISA assay results representing the average IL-10 concentration in CSF samples (n=5-6, mean±SEM, Kruskal Wallis test, followed by Dunn's posthoc, *p<0.05). FIG. 8D shows ELISA assay results representing the average TNFα concentration in CSF samples (n=5-6, mean±SEM, Kruskal Wallis test, followed by Dunn's posthoc, **p<0.01). GLED exposure resulted in increased levels of IL-10 and decreased levels of TNFα only in CSF.

GLED modulates cerebrospinal fluid (CSF) levels of inflammatory cytokines in male rats. Surgeries typically increase the levels of TNFα which shift the balance of cytokines to promote an inflammatory status. Additionally, IL-10 has analgesic effects in chronic pain conditions including inflammatory and neuropathic pain. To further elucidate the mechanism of GLED reversal of postoperative hypersensitivity, we investigated levels of inflammatory cytokines in the serum and in the cerebrospinal fluid (CSF) of elderly male rats. Animals received paw incision surgery and were exposed to either GLED or WLED. Animals exposed to GLED either had 0 or 4 days of pre-surgery exposure, as well as 2 days post-surgery exposure (FIG. 8A). At the end of exposure, blood and CSF were collected for quantification of anti-inflammatory IL-10 and pro-inflammatory TNFα by ELISA. In the serum, no significant differences in IL-10 levels were found between groups (FIG. 8B). Importantly, we were not able to detect TNFα in the serum with our ELISA assays. On the other hand, CSF IL-10 levels in the 6-day GLED group were significantly higher than that of the 6-day WLED group (FIG. 8C). The CSF IL-10 levels in the 2-day GLED group tended to be higher than that of the 6-day WLED group, although no significant differences were found (p=0.7931). Furthermore, CSF TNFα levels in the 6-day GLED group were significantly lower than that of the 6-day WLED group (FIG. 8D). CSF TNFα levels in the 2-day GLED group tended to be lower than that of the 6-day WLED group, although no significant differences were found (p=0.5785). Overall, these results show that a 6-day GLED exposure decreases levels of TNFα and increases levels of IL-10 in the CSF of elderly male rats, but not in the serum. In conjunction with increased levels of endogenous opioids specifically in the CSF, these results suggest that exposure to green light induces antinociception through central modulation rather than acting on the peripheral nervous system.

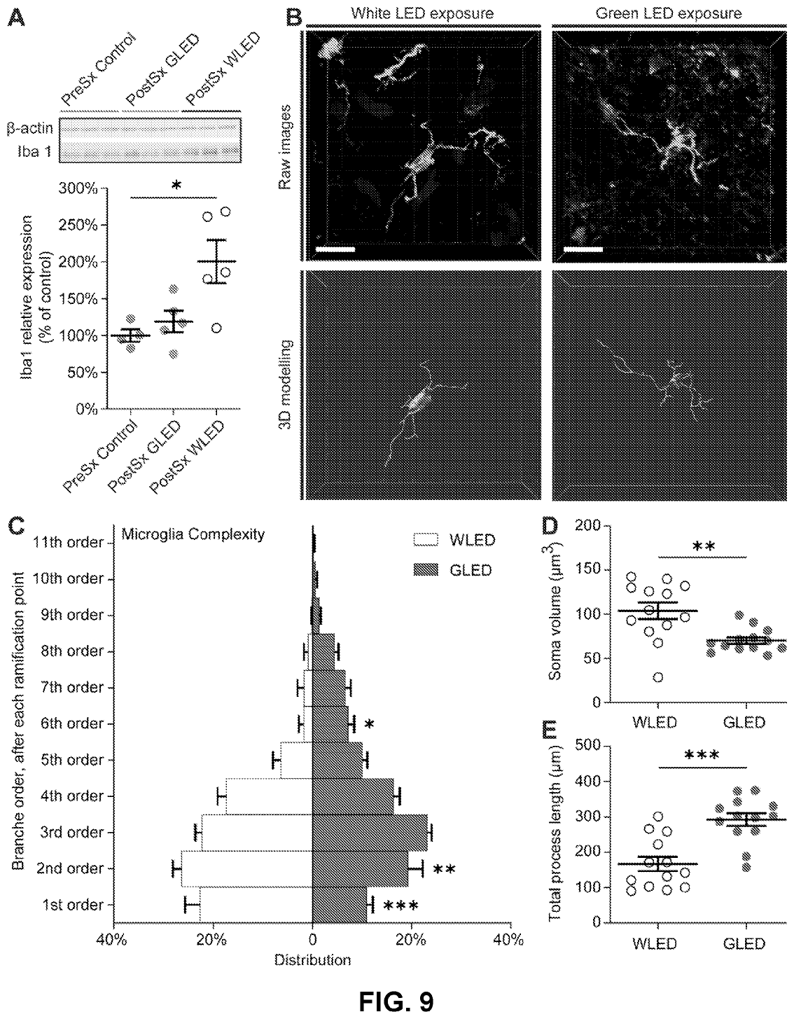

FIG. 9 shows that GLED exposure decreases microglial activation in male rats after surgery and provides an analysis of neuroinflammation status before and after exposure to white LED (WLED) or green LED (GLED). Male rats were exposed two days before and after paw incision surgery. FIG. 9A illustrates Iba1 expression (marker of microglial activation) before exposure (Preop. Control) and after surgery in both WLED and GLED groups (Postop. White and Postop. Green, respectively). Total lysates from the dorsal horn of the lumbar spinal cord were analyzed in western blot to evaluate Iba1 expression (n=4-5, mean±SEM, Kruskal Wallis, followed by Dunn's posthoc, *p<0.05). Surgeries resulted in increased Iba1 expression when rats were exposed to WLED but not GLED. FIG. 9B shows representative images of microglia after exposure to WLED or GLED and their respective 3D modeling. Scale bar=10 μm. FIG. 9C illustrates that, after modelling, microglia complexity was assessed by quantifying the number of branches of each order. Branches from the first order correspond to segments starting from the soma. Branches from the second order correspond to segments after the first ramification. Results illustrate distribution of branches in WLED and GLED conditions, at the end of exposure (n=13 microglia, mean±SEM, two-way ANOVA followed by Sidak's posthoc, *p<0.05, **p<0.01, ***p<0.001). FIG. 9D illustrates microglia soma volume after exposure to WLED or GLED (n=13 microglia per condition, mean±SEM, Mann Whitney test, **p<0.01). FIG. 9E illustrates microglia process length after exposure to WLED or GLED (n=13 microglia per condition, mean±SEM, Mann Whitney test, ***p<0.001). GLED exposure resulted in increased microglial complexity and smaller soma volume.

GLED reduces microglial activation in the spinal cord from male rats. Microglia are one of the main sources of TNFα in the central nervous system and regulate central inflammation. Furthermore, microglia play an essential function in regulating inflammation related to pain. Given their crucial role in pain, we sought to investigate if GLED exposure had any effects on microglial activation in the spinal cord that could be contributing to the induced antinociception. To initially evaluate any changes in microglial activation, total lysates of lumbar dorsal horns were analyzed by western blot for expression of Iba1, a marker of microglial activation. Iba1 expression in the post-surgery white group (the one exposed to WLED) was significantly higher than that of the pre-surgery control, indicating that paw incision surgery resulted in microglial activation (FIG. 9A). However, when comparing Iba1 expression between post-surgery GLED and pre-surgery Control groups, no significant differences were found, indicating that GLED exposure may attenuate microglial activation by paw incision surgery (FIG. 9A).

Microglia morphology reflects their activation states. As Iba1 expression is decreased in the dorsal horn of the spinal cord in males, we sought to further investigate the effects of GLED exposure on microglial morphology by 3D morphometric analyses. After image acquisition of microglia from male rats, 3D modeling was performed to analyze the complexity of microglial process branching (FIGS. 9B and 9C). Microglial complexity was assessed by analyzing the distribution of their processes in relation to branching points. First order branches referred to process segments between somas and first ramifications. Seventh order branches referred to process segments between the 6th and 7th branching points. This distribution analysis allowed us to have a clear visualization of microglia complexity and arborization. Exposure to GLED resulted in a less ramified arborization of microglia (FIG. 9C). Furthermore, soma volumes were significantly lower than that of the WLED group (FIG. 9D), and total length of processes were significantly higher in the GLED group (FIG. 9E). These changes in microglia morphology show that GLED exposure results in reduced microglial activation in elderly male rats.

Our data characterized the antinociceptive effect of GLED phototherapy on postoperative pain. GLED reversed hypersensitivity resulting from incisional surgery. Importantly, the hypersensitivity reversal heightened with increasing time of exposure. Furthermore, the effect was still observable even 3 days after GLED exposure termination. These results demonstrate that GLED possesses high potential as a post-operative pain management tool, characterized by a time-dependent and long-lasting effect.

In the elderly, opioid-derived side effects are further pronounced and raise the need to develop new approaches that can reduce opioid prescription. Our previous study revealed an increase in PENK mRNA expression after exposure to GLED. Thus, we evaluated the potential benefits of GLED to reduce opioid consumption. In male rats, GLED potentiated both mechanical and thermal opioid-induced analgesia. Paradoxically, we did not observe any potentiation in female rats when thermal hypersensitivity was evaluated. However, pain processing is different between sexes. For instance, estrogen and prolactin pathways may enhance central nervous system sensitization. Notably, studies conducted on human subjects revealed that GLED was analgesic in female patients.

To understand how GLED could potentiate opioid's effects, we explored different systems involved in pain modulation in male rats. Antinociceptive mechanisms rely in part on modulation of the endogenous opioid system. Hence, we first investigated whether GLED exposure could increase endogenous opioid levels or receptors. Our results showed an increase in β-endorphin and proenkephalin in the CNS in male rats, but expression of their receptors remained unchanged. Interestingly, we were not able to observe any differences of endogenous opioid levels in serum. This raises the possibility that GLED effect depends more on central mechanisms than peripheral pain modulation.

To gain insight into the mechanisms by which GLED could act centrally, we then delved into its potential effect on plasticity. Prolonged postoperative pain is related to central sensitization which is a major complication of surgeries. Glutamatergic receptors are key modulators of central sensitization, and blocking AMPA receptors result in reduced postoperative hypersensitivity both in rats and humans. Based on these observations, we analyzed GLED impact on glutamatergic receptors in male rats. Our results showed that GLED reduced phosphorylation and synaptic expression. Several studies have shown that activation and expression of AMPA receptors are increased in multiple pain models. Consequently, the decreased phosphorylation and synaptic expression of GluR1 explain how GLED could weaken transmission of acute pain signals in males, by modulating plasticity mechanisms in the spinal cord. Furthermore, studies suggest that spinal cord central sensitization shares common mechanisms with synaptic potentiation in the hippocampus. Our observations conclude to a decreased phosphorylation of Serine831 (S831), but not Serine845 (S845), after GLED exposure. In the hippocampus, S845 phosphorylation is crucial for both LTP and LTD, whereas S831 phosphorylation is required for LTP. This raises the hypothesis that GLED modulates plasticity by weakening synapses' ability to potentiate rather than inducing synaptic depression. Our results also demonstrate that GLED abates NMDA receptor synaptic expression and phosphorylation. NMDA receptor activation and phosphorylation are required for NMDA-dependent synapse potentiation, and blocking NMDA receptors reduces postoperative pain. By reducing synaptic expression of NMDA receptors in elderly males, GLED would reduce the synapse's capability to potentiate, thus weakening synaptic potentiation and pain signal transmission. Altogether, our results demonstrated how light stimulation modulates CNS plasticity in male rats.

Inflammation plays a key role in pain signaling and modulates glutamatergic receptor activity. TNFα increases glutamatergic receptor activity, whereas IL-10 decreases postsynaptic expression of AMPA receptors. Surgeries typically increase TNFα levels which shift the cytokine balance to promote an inflammatory status. Consequently, we measured serum and CSF levels of these cytokines in male rats, after surgery. GLED exposure increased IL-10 and decreased TNFα levels, specifically in the CSF. This suggests that decreasing TNFα levels may improve postoperative pain. IL-10 is also a key factor in postoperative pain reduction; overexpression of IL-10 decreases surgical inflammation and promotes tissue regeneration. As microglia are a major source of cytokines in the CNS, we also analyzed their activation following GLED exposure in elderly male rats. Microglia morphology is one of the best parameters to analyze their activation; increases in soma size, roundness, and simple arborization correspond to more active microglia. Our observations reveal that microglia are less activated following GLED exposure. Importantly, microglial modulation of nociceptive signals is different between male and female mice. Although the discrepancy of our results concerns thermal hypersensitivity, it is worth mentioning that microglia are not required for mechanical hypersensitivity in females. This observation raises the hypothesis that females are less affected by GLED due to its specific impact on microglia. In males, it is reasonable to assume that decreased levels of TNFα are in part due to microglial deactivation. Surgeries are associated with microglial activation, and their activation promotes TNFα release during postoperative neuroinflammation. Therefore, preoperative strategies to decrease microglia activation may result in less inflammatory mediator release and decreased pain. This raises the possibility that GLED exposure may be responsible for an improved inflammatory environment, lowering pain sensitivity and promoting healing.

Altogether, our results suggest that GLED improves postsurgical pain in male and female rats by acting on multiple systems. On the one hand, GLED stimulates endogenous opioid release, lowering exogenous opioid needs after surgery. On the other hand, GLED modulates CNS plasticity by reducing expression of receptors involved in pain transmission, while providing a better inflammatory environment. Importantly, all these systems are interconnected. Increasing evidence shows that TNFα is a key modulator of analgesia, and a reciprocal connection between this cytokine and the endogenous opioid system is evident. However, TNFα possesses a paradoxical effect on endogenous opioid release; TNFα inhibits morphine analgesia after acute administration, whereas administration of morphine inhibits TNFα production. Cytokines not only modulate the opioid system, but also regulate microglia activity, which is crucial for neuronal transmission. Indeed, activation of microglia induces an increase of spontaneous excitatory postsynaptic currents, and recent studies suggest that microglia-derived BDNF activates Src kinase which in turn increases NMDA receptor transmission in rat pain models. Importantly, increasing microglial activation alters LTP, which requires AMPA receptors, and elevated levels of TNFα stimulates AMPA receptor endocytosis.

Overall, GLED modulates three different systems that are interconnected, providing a decreased inflammatory environment that can act on synaptic transmission. Light exposure could modulate inflammation and plasticity: exposing mice to flickering lights at 40 Hz drives neural activity and recruits microglia. Light flickering stimulation in Alzheimer's disease models also shown reduced inflammation and preservation of neuronal and synaptic density across multiple brain areas.

Managing inflammation in the elderly has become crucial and gave rise to the concept of “senoinflammation” or “inflammaging”, chronic inflammation related to aging. This chronic inflammation exacerbates aging processes and age-related chronic diseases. Given that GLED exposure reduces neuroinflammation, this new therapy may offer a more appropriate way to control inflammation-related pain in the elderly. A study suggested that migraine photophobia could originate in cone-driven retinal pathways and be tuned in thalamic neurons outside the main visual pathway. Strong evidence also suggests that suprachiasmatic nucleus (SCN), site of the master circadian clock, regulates inflammatory responses and monochromatic light can affect cognitive processes almost instantaneously. These effects are mediated by a melanopsin-based photoreceptor system, which is known to transmit irradiance signals to the SCN. GLED exposure could modulate SCN functions, thus affecting circadian rhythms that are highly involved in regulating inflammatory processes related to pain condition. Furthermore, a circadian rhythm of endogenous opioids and β-endorphin levels are highest in the morning. By modulating functions of the SCN and circadian rhythms, GLED could affect inflammation and endogenous opioid release. Consecutively to microglia deactivation, decreased levels of TNFα and increased levels of IL-10 may modulate pain signaling transmission through acting on glutamatergic receptors. Capitalizing on several mechanisms to control pain may have a better outcome for postoperative pain in the elderly. Furthermore, this therapy may reduce opioid requirements to control postoperative pain, and such a strategy may be especially crucial for elderly patients. Given the noninvasive nature of GLED, it would be relatively safe to translate and clinically implement this innovative therapy. Providing all patients, especially the elderly, with better post-surgical pain control while decreasing opioids requirement may facilitate discharge from the hospitals, early mobility, and fewer comorbidities associated with uncontrolled post-operative pain and opioid consumption.

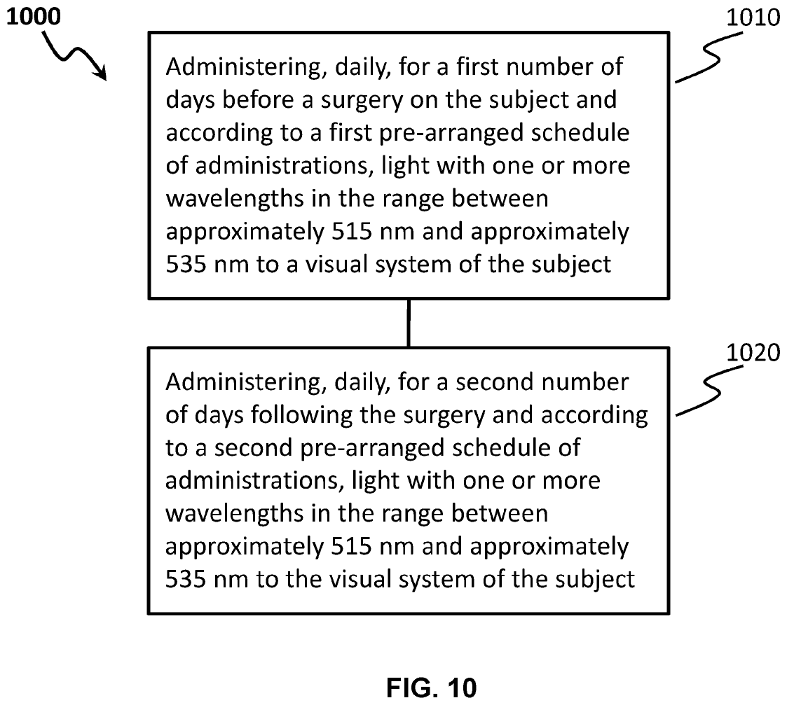

FIG. 10 shows a flow diagram of an example embodiment of a method 1000 of reducing post-operative pain of a subject according to the disclosed technology. Step 1010 of the method 1000 includes administering, daily, for a first number of days before a surgery on the subject and according to a first pre-arranged schedule of administrations, light with one or more wavelengths in the range between approximately 515 nm and approximately 535 nm to a retina of the subject. Step 1020 of the method 1000 includes administering, daily, for a second number of days following the surgery and according to a second pre-arranged schedule of administrations, light with one or more wavelengths in the range between approximately 515 nm and approximately 535 nm to the retina of the subject.

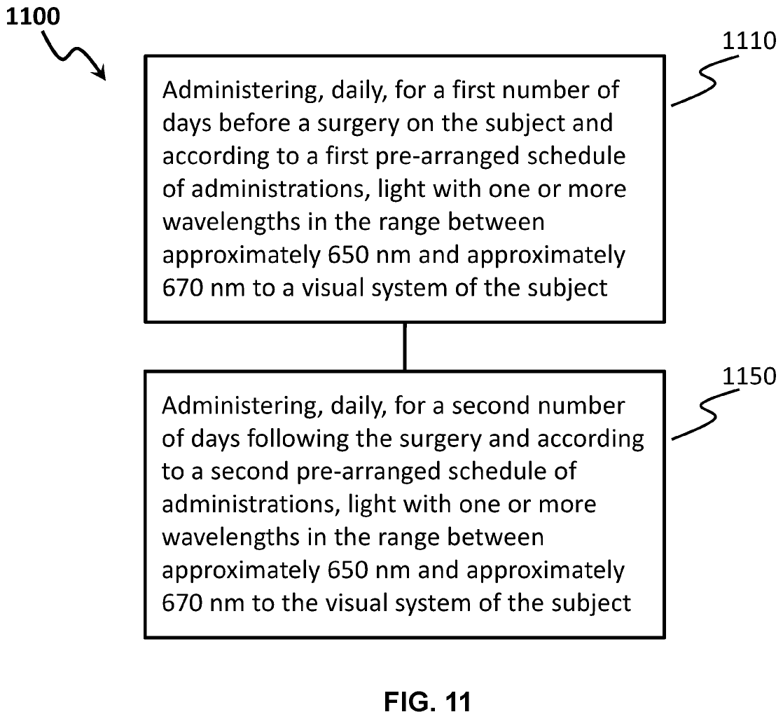

FIG. 11 shows a flow diagram of an example embodiment of a method 1100 of enhancing pain sensitivity of a subject according to the disclosed technology. Step 1110 of the method 1100 includes administering, daily, for a first number of days before a surgery on the subject and according to a first pre-arranged schedule of administrations, light with one or more wavelengths in the range between approximately 650 nm and approximately 670 nm to a retina of the subject. Step 1120 of the method 1100 includes administering, daily, for a second number of days following the surgery and according to a second pre-arranged schedule of administrations, light with one or more wavelengths in the range between approximately 650 nm and approximately 670 nm to the retina of the subject.

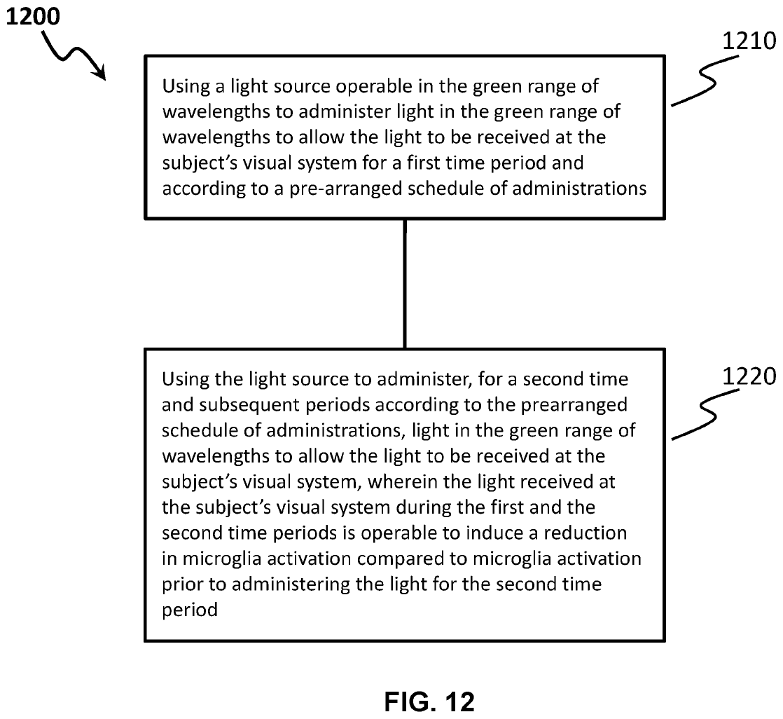

FIG. 12 shows a flow diagram of an example embodiment of a method 1200 for reducing microglial activation in a subject according to the disclosed technology. Step 1210 of the method 1200 includes using a light source operable in the green range of wavelengths to administer light in the green range of wavelengths to allow the light to be received at the subject's visual system (e.g., subject's pupil) for a first time period and according to a pre-arranged schedule of administrations. In Step 1220, the light source is used to administer, for a second time and subsequent periods according to the prearranged schedule of administrations, light in the green range of wavelengths to allow the light to be received at the subject's visual system. The light received at the subject's pupil during the first and the second time periods is operable to induce a reduction in microglia activation compared to microglia activation prior to administering the light for the second time period. In one example embodiment, the light received at the subject's visual system is operable to regulate inflammation by inducing the reduction microglia activation. In another example embodiment, the first time period occurs prior to a surgery on the subject, and the second time period occurs after the surgery on the subject. In another example embodiment, the light received at the subject's pupil is operable to reduce release of at least tumor necrosis factor alpha (TNFα) during postoperative neuroinflammation.

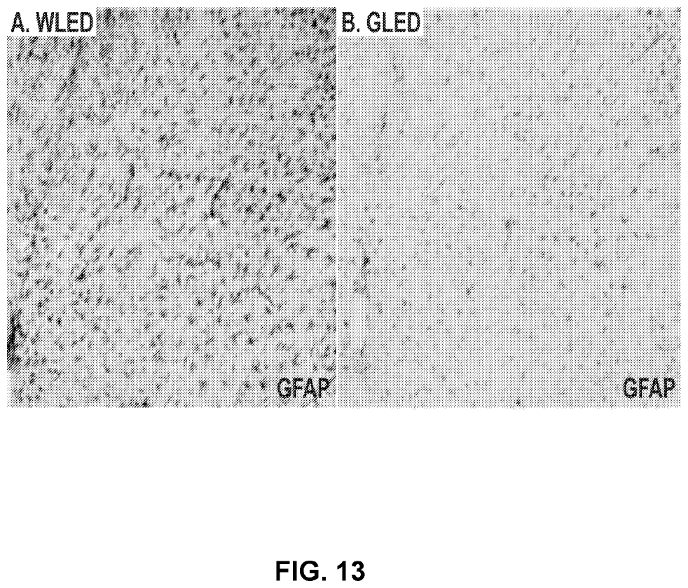

In addition to microglia inactivation, GLED also decreases astrogliosis. Reactive astrocytes respond according to different signals and impact neuronal function in multiple medical conditions. We found out that GLED decreased pain associated with HIV infection in rats. HIV infection induces changes in the molecular expression and morphology of astrocytes in the central nervous system, characteristic of their reactive state. In an experiment, we initially infected rats with gp-120 HIV envelope protein in the spinal cord of rats. After the rats developed thermal and mechanical hypersensitivity, they were divided into two groups. One group of rats received GLED, and the other group received WLED. Light exposure was for 5 days for 8 hours a day. Administrations of WLED and GLED were carried out for a first period of time according to a predetermined schedule. The group that received GLED was no longer sensitive to thermal or mechanical stimuli, suggesting that GLED reversed the hypersensitivity associated with HIV infection. On the other hand, the rats exposed to WLED remained hypersensitive to thermal and mechanical stimuli. The rats were then sacrificed, and the spinal cords were collected. We then utilized immunohistochemistry with antibodies directed at the GFAP (upregulated in reactive astrocytes). The immunohistochemistry revealed that GLED decreased astrogliosis in the dorsal horn of the spinal cord. On the other hand, WLED exposure resulted in an increased number of reactive astrocytes. FIG. 13 illustrates the effects of WLED (Panel A) and GLED (Panel B) on astrocyte reactivity in HIV-infected rats, where darker staining indicates increased reactivity of astrocytes. More specifically, FIG. 13 shows representative micrographs of 10-μm sections of the dorsal horn of the spinal cord from either control in Panel A, or GLED-exposed rats, in Panel B, immunostained for glial fibrillary acid protein (GFAP). As evident from the figure, GLED exposure decreases astrocyte activation.

The following are further details of an example method for the above noted animal perfusion and spinal cord collection. To collect spinal cords from Sprague-Dawley male rats, animals were anesthetized with 1-5% isoflurane delivered in 02 and then perfused with phosphate-buffered saline (PBS) and 4% paraformaldehyde (PFA). Single 4 cm lateral incision was made through integument and abdominal wall just beneath rib cage. A cut was then made through rib cage up to the clavicle. A 20-gauge needle was inserted through left ventricle into ascending aorta. Approximately 30 mL of PBS and 30 mL of 4% PFA were injected through the heart at a steady flow rate. After fixation, rats were decapitated, and lumbar spinal cords were collected by hydraulic extrusion. Spinal cords were postfixed in 4% PFA for 2 hours and then immersed in 30% sucrose for 24 hours. Spinal cords were then flash frozen into Tissue-Tek and stored at −80° C. 15 μm frozen sections were sliced using Cryostar NX50.

Astrocytes immunostainings were conducted as follows: slices were permeabilized in 0.3% Triton-X-100 with 1% BSA diluted in PBS, and proteins of interest were stained overnight at 4° C. in the same buffer with antibodies directed against GFAP. After primary probing, slices were rinsed and incubated for 2 hours at room temperature with appropriate fluorescent conjugated secondary antibodies (Jackson Immunoresearch). Slices were mounted with FluorSave reagent (Millipore #345789).

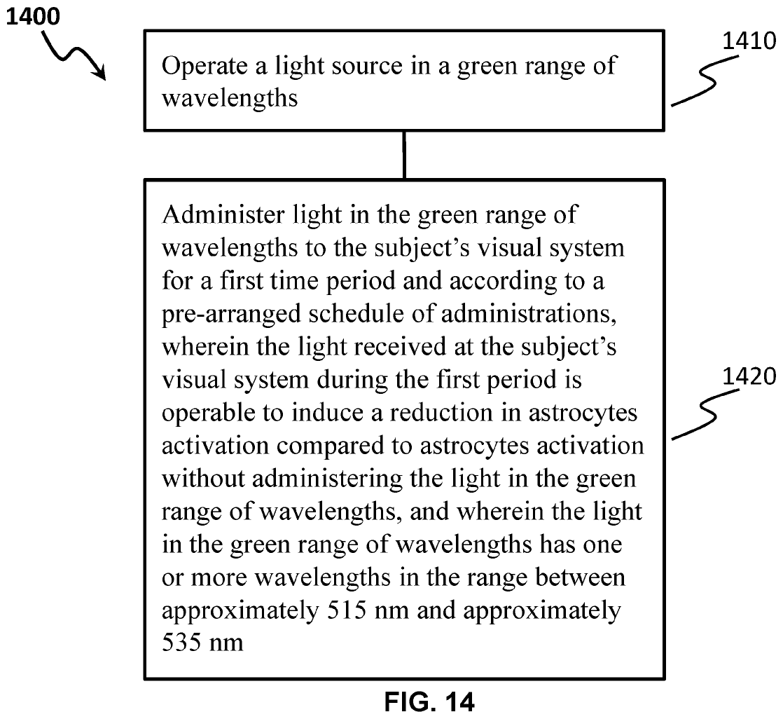

FIG. 14 illustrates a set of operations that can be carried for reducing astrogliosis according to an example embodiment. At 1410, a light source in a green range of wavelengths is operated. At 1420, light in the green range of wavelengths is administered to the subject's visual system for a first time period and according to a pre-arranged schedule of administrations. The light received at the subject's visual system during the first period is operable to induce a reduction in astrocytes activation compared to astrocytes activation without administering the light the green range of wavelengths. The light in the green range of wavelengths has one or more wavelengths in the range between approximately 515 nm and approximately 535 nm. In one example embodiment, the light received at the subject's visual system is operable to reverse changes in molecular expression or morphology of astrocytes indicative of a reactive state of the astrocytes. In another example embodiment, the changes in the molecular expression or morphology of astrocytes is induced due to an infection. In one example embodiment, the infection is associated with a human immunodeficiency virus. In another example embodiment, the light received at the subject's visual system is operable to reduce pain. In yet another example embodiment, the first time period for administering the light in the green range of wavelengths is a first number of hours less than equal to eight, and the pre-arranged schedule of administrations consists of administrations over a plurality of consecutive days.

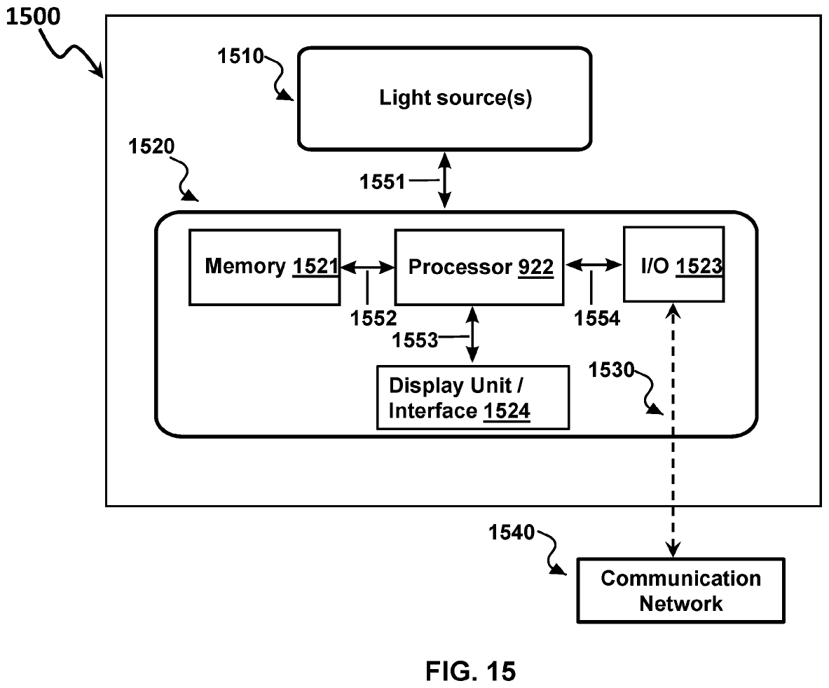

FIG. 15 shows a schematic diagram of an example embodiment of a device 1500 for modulating pain sensitivity of a subject according to the disclosed technology. The device 1500 includes one or more light sources having emission spectra within a range from approximately 515 nm to approximately 535 nm associated with green light or a range from approximately 650 nm to approximately 670 nm associated with red light (element 1510 in FIG. 15). The device 1500 also includes a controller 1520 configured to control at least one of: intensity of light emission or duration of light emission of the one or more light sources 1510. In some embodiments, the controller 1520 can include a memory 1521, a processor 1522, and a data input/output (I/O) interface 1523. The memory 1521 of the controller 1520 can store a code which, when executed by the processor 1522 can make the controller 1520 control at least one of: intensity of light emission or duration of light emission of a light source in the one or more light sources 1510. In some embodiments, device 1500 can also include a display or interface unit 1524 that can allow a user of the device 1500 enter commands for the device 1500 and/or allow the device 1500 provide information (in, e.g., a graphical or a text form) to the user. In some embodiments, the I/O 1523 of the controller 1520 of the device 1500 can be connected to a communication network (e.g., Internet) 1540 via a communication link 1530. Arrows 1551, 1552, 1553, and 1554 in FIG. 15 illustrate communication links between different elements of the device 1500. According to some example embodiments, the device 1500 can be one of: goggles, ski goggles, a pair of eyeglasses, or a contact lens.

U.S. Pat. No. 10,857,382, which is incorporated by reference herein, includes additional information and examples related to methods, systems and devices that can complement the disclosed technology.

The following examples may be preferable features of some implementations of the disclosed technology.

An example aspect of the embodiments disclosed in this patent document (example E1) relates to a method of reducing post-operative pain in an animal, including man, involving optic exposure to green light. Example E2 includes the method of example E1 wherein wavelengths of the light range between 515 nm and 535 nm. Example E3 includes the method of example E1 wherein light on a selected wavelength is applied between eight weeks prior, up to the time of surgery, and following the surgery for up to twelve weeks. Example E4 includes the method of example E1 wherein the length of daily exposure is between 10 min and 16 hrs.

Another example aspect of the embodiments disclosed in this patent document (example E5) relates to a method of modulating central nervous system inflammation via direct optic exposure to green light in an animal, including man, as outlined in examples E1-E4. Example E6 includes the method of example E5 wherein the green light decreases inflammatory mediator levels and enhances anti-inflammatory mediator levels. Example E7 includes the method of example E5 wherein the green light increases an IL-10 level and decreases a TNFα level.

Yet another example aspect of the embodiments disclosed in this patent document (example E8) relates to a method of modulating neuronal plasticity in an animal, including man, involving optic exposure to green light as in examples E1-E4.

An example aspect of the embodiments disclosed in this patent document (example E9) relates to a method of decreasing activation of central nervous system (CNS) immune cells in an animal, including man, involving optic exposure to green light as in examples E1-E4.

Another example aspect of the embodiments disclosed in this patent document (example E10) relates to a method of reducing opioid and analgesic needs via exposure of an animal, including man, to green light as outlined in examples E1-E4.

Yet another example aspect of the embodiments disclosed in this patent document (example E11) relates to a method of reducing manifestations of neurodegenerative conditions in an animal, including man, involving optic exposure to green light as in examples E1-E4. Example E12 includes the method of the example E11, wherein neurodegenerative conditions include at least one of: presenile (Alzheimer) dementia, senile dementia, or vascular dementia.

An example aspect of the embodiments disclosed in this patent document (example E13) relates to a method of potentiation—including extending the time and magnitude of efficacy as well as enhancing the potency—of pharmacologic analgesic agents in an animal, including man, via optic exposure to green light as outlined in examples E1-E4.

Another example aspect of the embodiments disclosed in this patent document (example E14) relates to a method of potentiation (including extending the time and magnitude of efficacy as well as enhancing the potency) of pharmacologic analgesic agents in an animal, including man, via modulating the long term potentiation process through alteration of NMDA and AMPA receptors via optic exposure to green light as outlined in examples E1-E4.

Yet another example aspect of the embodiments disclosed in this patent document (example E15) relates to a method of enhancing pain sensitivity via exposure of an animal, including man, to red light. Example E16 includes the method of example E15 wherein wavelengths of the light range between 650 nm and 670 nm. Example E17 includes the method of example E15 wherein light on a selected wavelength is applied between eight weeks prior, up to the time of surgery, and following the surgery for up to twelve weeks. Example E18 includes the method of example E15 wherein the length of daily exposure is between 10 min and 16 hrs.

An example aspect of the embodiments disclosed in this patent document (example E19) relates to a system for exposing an animal, including man, to light, as outlined in the above examples E1-E18, comprising light exposure means and a control system regulating time and/or intensity (e.g., dose) of the exposure.

Another example aspect of the embodiments disclosed in this patent document (example E20) includes a device for exposing an animal, including man, to light as outlined in the examples E1-E19.

An example aspect of the technology disclosed in this patent document (example E21) relates to a method of reducing post-operative pain of a subject, comprising: administering, daily, for a first number of days before a surgery on the subject and according to a first pre-arranged schedule of administrations, light with one or more wavelengths in the range between approximately 515 nm and approximately 535 nm to a visual system (e.g., retina) of the subject; and administering, daily, for a second number of days following the surgery and according to a second pre-arranged schedule of administrations, light with one or more wavelengths in the range between approximately 515 nm and approximately 535 nm to the visual system of the subject, wherein the light is administered for a time period during each day of the light administration. Example E22 includes the method of example E21 wherein the first number of days is 365 or less. Example E23 includes the method of example E21 wherein the second number of days is 365 or less. Example E24 includes the method of example E21 wherein the time period is between 1 minute and 24 hours. Example E25 includes the method of example E21 wherein the administering comprises administering light of 0.001 lux to 1000 lux. Example E26 includes the method of example E21 wherein the method further comprises administering an opioid or non-opioid pain relief medication to the subject after the surgery. Example E27 includes example E26 wherein the opioid or non-opioid pain relief medication is administered at a reduced dose compared to a standard dose of the pain relief medication for the subject. Example E28 includes the method of example E21 wherein for at least two days for which said administering is performed during them there is at least one day between the two days during which no said administering is performed. Example E29 includes the method of example E21 wherein the administering includes contacting an eye of the subject with a material that allows light between approximately 515 nm and approximately 535 nm to enter the visual system of the subject. Example E30 includes example E29 wherein the material is a material that is configured to filter out light of a wavelength that is not between approximately 515 nm and approximately 535 nm.

Another example aspect of the technology disclosed in this patent document (example E31) relates to a method of enhancing pain sensitivity of a subject, comprising: administering, daily, for a first number of days before a surgery on the subject and according to a first pre-arranged schedule of administrations, light with one or more wavelengths in the range between approximately 650 nm and approximately 670 nm to a visual system of the subject; and administering, daily, for a second number of days following the surgery and according to a second pre-arranged schedule of administrations, light with one or more wavelengths in the range between approximately 650 nm and approximately 670 nm to the visual system of the subject, wherein the light is administered for a time period during each day of the light administration. Example E32 includes the method of example E31 wherein the first number of days is 365 or less. Example E33 includes the method of example E31 wherein the second number of days is 365 or less. Example E34 includes the method of example E31 wherein the time period is between 1 minute and 24 hours. Example E35 includes the method of example E31 wherein the administering comprises administering light of 0.001 lux to 1000 lux. Example E36 includes the method of example E31 wherein for at least two days for which said administering is performed during them there is at least one day between the two days during which no said administering is performed. Example E37 includes the method of example E31 wherein the administering includes contacting an eye of the subject with a material that allows light between approximately 650 nm and approximately 670 nm to enter the visual system of the subject. Example E38 includes example E37 wherein the material is a material that is configured to filter out light of a wavelength that is not between approximately 650 nm and approximately 670 nm.

Another example aspect of the technology disclosed in this patent document (example E39) relates to a device for modulating pain sensitivity of a subject, comprising: one or more light sources having emission spectra within a range from approximately 515 nm to approximately 535 nm associated with green light or a range from approximately 650 nm to approximately 670 nm associated with red light; and a controller configured to control at least one of: intensity of light emission or duration of light emission of the one or more light sources, wherein the device is configured to allow administration of the green light or the red light to a person wearing the device. Example E40 includes the device of example E39 wherein the device is one of: goggles, ski goggles, a pair of eyeglasses, or a contact lens.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

It is understood that the various disclosed embodiments may be implemented individually, or collectively, using devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. For example, the processor may be configured to receive electrical signals or information from the disclosed sensors (e.g., CMOS sensors), and to process the received information to produce images or other information of interest.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes

Only a few implementations and examples are described and other implementations, enhancements and variations can be made based on what is described and illustrated in this patent document.

1. A method of reducing post-operative pain of a subject, comprising:
administering, daily, for a first number of days before a surgery on the subject and according to a first pre-arranged schedule of administrations, light with one or more wavelengths in the range between approximately 515 nm and approximately 535 nm to a visual system of the subject; and
administering, daily, for a second number of days following the surgery and according to a second pre-arranged schedule of administrations, light with one or more wavelengths in the range between approximately 515 nm and approximately 535 nm to the visual system of the subject,
wherein the light is administered for a time period during each day of the light administration.

2. The method of claim 1, wherein the first number of days is 56 or less, the second number of days is 84 or less, and the time period is between 1 minute and 24 hours.

3. (canceled)

4. (canceled)

5. The method of claim 1, wherein the administering comprises administering light of 0.001 lux to 1000 lux.

6. The method of claim 1, further comprising administering an opioid or non-opioid pain relief medication to the subject after the surgery, wherein said opioid or non-opioid pain relief medication is administered at a reduced dose compared to a standard dose of the pain relief medication for the subject.

7. (canceled)

8. The method of claim 1, wherein for at least two days for which said administering is performed during them there is at least one day between the two days during which no said administering is performed.

9. The method of claim 1, wherein said administering includes contacting an eye of the subject with a material that allows light between approximately 515 nm and approximately 535 nm to enter the visual system of the subject, and wherein the material is a material that is configured to filter out light of a wavelength that is not between approximately 515 nm and approximately 535 nm.

10. (canceled)

11. A method of enhancing pain sensitivity of a subject, comprising:
administering, daily, for a first number of days before a surgery on the subject and according to a first pre-arranged schedule of administrations, light with one or more wavelengths in the range between approximately 650 nm and approximately 670 nm to a visual system of the subject; and
administering, daily, for a second number of days following the surgery and according to a second pre-arranged schedule of administrations, light with one or more wavelengths in the range between approximately 650 nm and approximately 670 nm to the visual system of the subject,
wherein the light is administered for a time period during each day of the light administration.

12. The method of claim 11, wherein the first number of days is 56 or less, the second number of days is 84 or less, and the time period is between 1 minute and 24 hours.

13. (canceled)

14. (canceled)

15. The method of claim 11, wherein the administering comprises administering light of 0.001 lux to 1000 lux.

16. The method of claim 11, wherein for at least two days for which said administering is performed, there is at least one day between the at least two days during which no said administering is performed.

17. The method of claim 11, wherein said administering includes contacting an eye of the subject with a material that allows light between approximately 650 nm and approximately 670 nm to enter the visual system of the subject, wherein the material is a material that is configured to filter out light of a wavelength that is not between approximately 650 nm and approximately 670 nm.

18. (canceled)

19. A method for reducing microglial activation in a subject, comprising:
using a light source operable in a green range of wavelengths to administer light in the green range of wavelengths to allow the light to be received at the subject's visual system for a first time period and according to a pre-arranged schedule of administrations; and
using the light source to administer, for a second time and subsequent periods according to the prearranged schedule of administrations, light in the green range of wavelengths to allow the light to be received at the subject's visual system;
wherein the light received by the subject's visual system during the first and the second time periods is operable to induce a reduction in microglia activation compared to microglia activation prior to administering the light for the second time period.

20. The method of claim 19, wherein the light received at the subject's visual system is operable to regulate inflammation by inducing the reduction microglia activation.

21. The method of claim 19, wherein the first time period occurs prior to a surgery on the subject, and the second time period occurs after the surgery on the subject.

22. The method of claim 21, wherein the light received at the subject's visual system is operable to reduce release of at least tumor necrosis factor alpha (TNFα) during postoperative neuroinflammation.

23. (canceled)

24. (canceled)

25. A method for reducing astrogliosis in a subject, comprising:
operating a light source in a green range of wavelengths; and
administering light in the green range of wavelengths to the subject's visual system for a first time period and according to a pre-arranged schedule of administrations;
wherein the light received at the subject's visual system during the first period is operable to induce a reduction in astrocytes activation compared to astrocytes activation without administering the light in the green range of wavelengths, and wherein the light in the green range of wavelengths has one or more wavelengths in the range between approximately 515 nm and approximately 535 nm.

26. The method of claim 25, wherein the light received at the subject's visual system is operable to reverse changes in the molecular expression or morphology of astrocytes indicative of a reactive state of the astrocytes.

27. The method of claim 26, wherein the changes in molecular expression or morphology of astrocytes are induced due to an infection, wherein the infection is associated with a human immunodeficiency virus.

28. (canceled)

29. The method of claim 25, wherein the light received at the subject's visual system is operable to reduce pain, wherein the first time period is a first number of hours less than equal to eight, and the pre-arranged schedule of administrations consists of administrations over a plurality of consecutive days.

30. (canceled)
